(19) United States
(12) Patent Application Publication
(10) Pub. No.: US 2023/0235900 A1
(43) Pub. Date: Jul. 27, 2023

(54) TWO STAGE AIR PURIFICATION SYSTEM FOR ENCLOSED LOCATIONS

(71) Applicant: KNORR BRAKE COMPANY LLC, Westminster, MD (US)

(72) Inventors: Richard Stelmach, Hanover, PA (US); Justin Jones, Thurmont, MD (US); Tyler Bowie, Westminster, MD (US); Jeremy Lowe, Westminster, MD (US)

(73) Assignee: KNORR BRAKE COMPANY LLC, Westminster, MD (US)

(21) Appl. No.: 18/010,705

(22) PCT Filed: Jun. 15, 2021
(86) PCT No.: PCT/US2021/037342

§ 371 Date: Dec. 15, 2022

Related U.S. Application Data
(63) Continuation-in-part of application No. 17/195,867, filed on Mar. 9, 2021, now Pat. No. 11,046,153.
(60) Provisional application No. 63/039,622, filed on Jun. 16, 2020.

(51) Int. Cl. F24F 8/192; F24F 8/30; F24F 8/80; F24F 11/88; B03C 3/12; B03C 3/09

(52) U.S. Cl. CPC F24F 8/192; F24F 8/30; F24F 8/80; F24F 11/88; B03C 3/12; B03C 3/09; A61L 9/22

(57) ABSTRACT

An air purification system for use in a given location to remove or destroy harmful pathogens in the air. The air purification system has a housing with an inlet, an outlet, and a passageway extending between the inlet and the outlet. An intense field generator and filter is used to charge any particles in the air and remove them from the air flow. Finally, a dielectric barrier discharge unit having a high voltage electrode coupled to a dielectric barrier and a ground electrode spaced apart from the high voltage electrode is used to form a low temperature plasma chamber is in communication with the passageway so that the ions created in the plasma chamber will attach to and destroy any remaining particles in the air flow.

### BACKGROUND OF THE INVENTION

### 1. Field of the Invention

The present invention relates to air treatment systems and, more specifically, to an air purification system that can reliably eliminate airborne pathogens in a given location.

### 2. Description of the Related Art

The current global COVID-19 pandemic has revealed the need for systems that can address airborne pathogens including viruses. For example, enclosed locations including rooms, building, and even vehicle such as those used for public transportation must remain safe to remain open during a pandemic or to be placed back into use after quarantining periods have ended and governments allow reopening. Current cleaning methods, such as ultraviolet (UV) radiation of surfaces can provide significant improvements to reduce spread of contagions, but there is still vast room for improvement. One significant risk point is the inability of conventional HVAC and air treatment systems to effectively filter out airborne viruses, including COVID-19.

### BRIEF SUMMARY OF THE INVENTION

The present invention provides an air purification system for use in an enclosed location that can reliably remove or destroy harmful pathogens. More specifically, the air purification system comprises a housing having an inlet, an outlet, and a passageway extending between the inlet and the outlet within the housing to define an air flow pathway. At least one fan is positioned in the housing to create and maintain a pressure differential along the passageway such that air can flow into the inlets, through the passageway, and out of the outlet along the air flow pathway. An intense field unit is coupled to the inlet, wherein the intense field unit comprises an intense field generator having a series of openings formed therethrough and a corresponding series of electrodes positioned in each of the series of openings so that a tip of each electrode extends into a center of each opening respectively, and an intense field dielectric filter having a plurality of channels formed therethrough and aligned with the openings of the intense field generator, wherein each channel is defined by a first surface comprising a first electrode and a second surface opposing the first electric and comprises a second electrode, and wherein the first electrode and the second electrode are encompassed by a dielectric material. A dielectric barrier discharge unit having a high voltage electrode coupled to a dielectric barrier and a ground electrode spaced apart from the high voltage electrode to define a low temperature plasma discharge chamber has the discharge chamber is in communication with the passageway to treat the air in the passageway with a plasma discharge. A first power source is coupled to the intense field generator to apply a first voltage to the tip of each electrode and to an edge of each opening that is sufficient to create a corona discharge therebetween. The voltage applied to the tip of each electrode and an edge of each opening is about 8000 volts of direct current. A second power source is coupled to the intense field dielectric filter to apply a second voltage to the first electrode and the second electrode. The second voltage is 24 volts of direct current. The channels are configured to result in a pressure drop in the air flow path of less than about 30 Pascals

The present invention also includes a method of purifying the air in a location. The first step is positioning an air treatment unit in the location, wherein the air treatment unit includes a housing having an inlet, and an outlet, and a passageway extending between the inlet and the outlet to define an air flow pathway, at least one fan positioned in the housing in the passageway, an intense field dielectric unit associated with the inlet, and a dielectric barrier discharge unit in communication with the passageway. In another step, the fan is operated to create and maintain a pressure differential along the passageway so that air flows from the location into the inlet, along the passageway, and out of the outlet into the location. The intense field generator is powered to create a corona discharge. The intense field filter is powered to capture any particles in the air that flows through the passageway that are charged by the corona discharge. The dielectric barrier discharge unit is powered to emit low temperature plasma into the passageway.

### BRIEF DESCRIPTION OF THE SEVERAL VIEWS OF THE DRAWING(S)

The present invention will be more fully understood and appreciated by reading the following Detailed Description in conjunction with the accompanying drawings, in which:

FIG. 1 is a perspective view of an air treatment system for an enclosed location according to the present invention;

FIG. 2 is a schematic of an air treatment system for an enclosed location according to the present invention;

FIG. 3 is a schematic of a two-phase air purification approach for an air treatment system according to the present invention;

FIG. 4 is a schematic of an intense field dielectric phase of an air treatment system according to the present invention;

FIG. 5 is a schematic of an intense field dielectric generator according to the present invention;

FIG. 6 is a schematic of an intense field dielectric filter according to the present invention;

FIG. 7 is a schematic of a microchannel of an intense field dielectric filter according to the present invention;

FIG. 8 is a perspective view of a microchannel of an intense field dielectric filter according to the present invention;

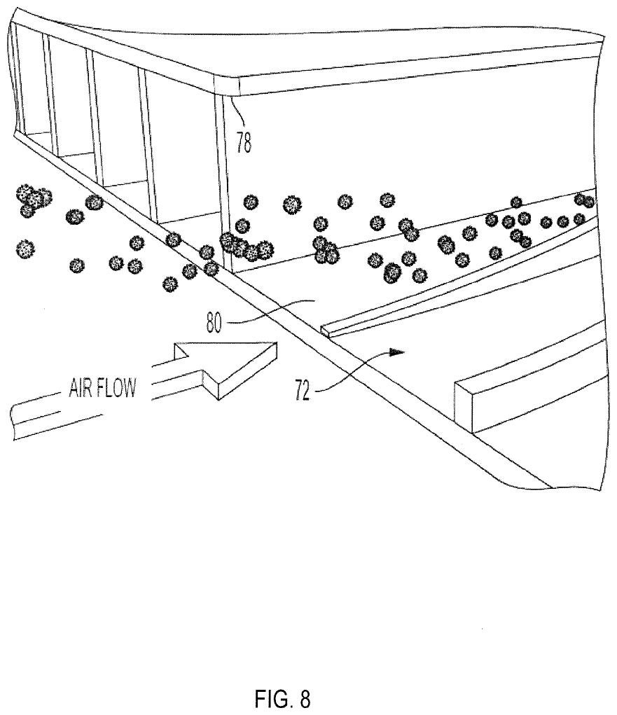

FIG. 9 is a schematic of dielectric barrier discharge phase of an air treatment system according to the present invention;

FIG. 10 is a schematic of an alternative arrangement for a dielectric barrier discharge unit according to the present invention.

### DETAILED DESCRIPTION OF THE INVENTION

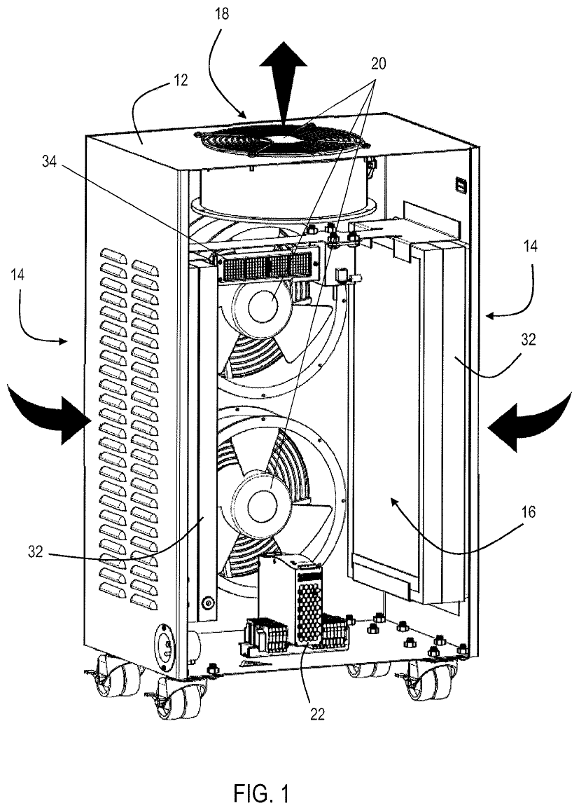

Referring to the figures, wherein like numeral refer to like parts throughout, there is seen in FIG. 1 an air treatment system 10 for eliminating airborne pathogens from an enclosed location. Air treatment system 10 generally comprises a housing 12 having opposing air inlets 14 that can withdraw air from an enclosed location, a passageway 16 into which air passes for treatment positioned between inlets 14 generally comprises an enclosed space within housing 12, and an air outlet 18 for returning purified or treated air from passageway 16 to the enclosed location. Air treatment system 10 includes one or more fans 20 for creating and maintaining a pressure differential along passageway 16 such that air flows into inlet 14, through passageway 16, and out of outlet 18 to define a complete air flow pathway 24. Air treatment system 10 additionally includes one or more power sources 22 that can connect to and transform local power (such 110/220 volt building supply) into the appropriate voltage for each element of the air treatment system 10, as explained below. It should be recognized that other power sources, including rechargeable batteries, may be used.

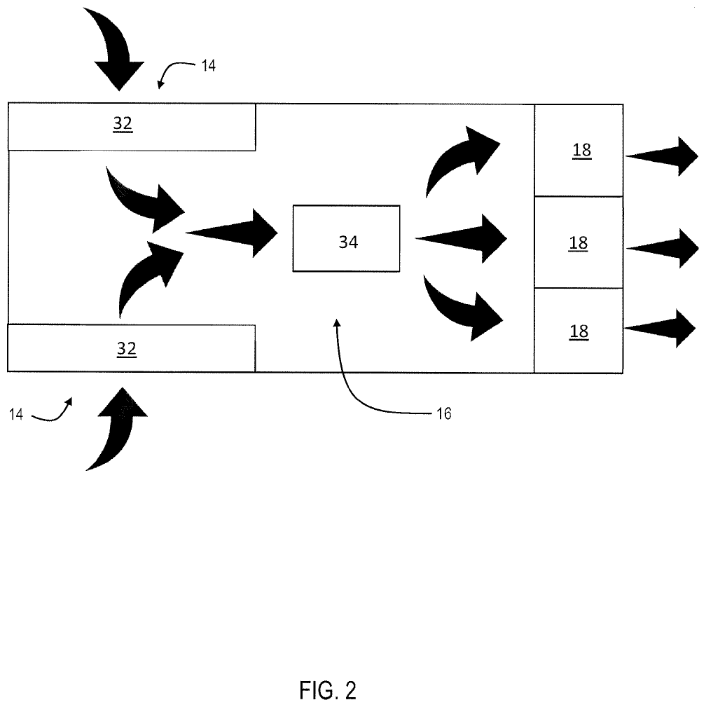

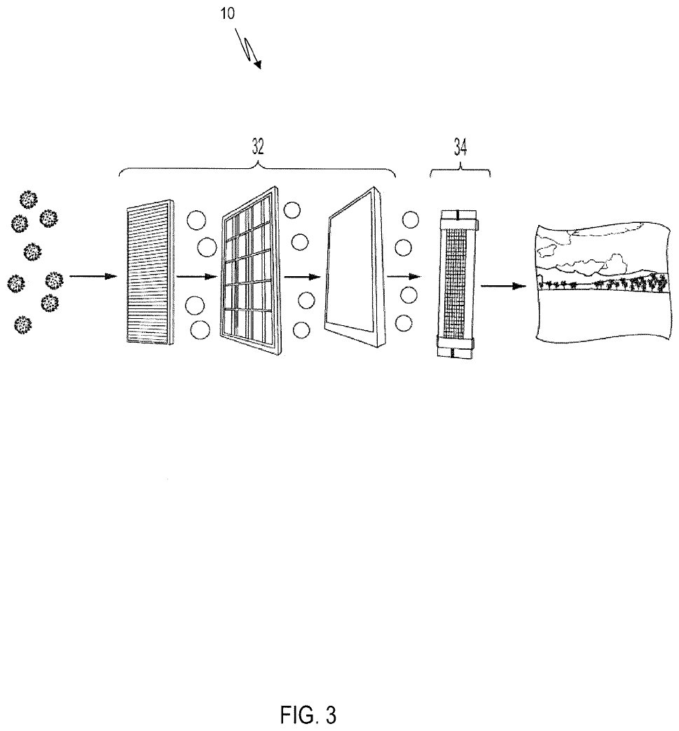

Referring to FIGS. 2 and 3, air treatment system 10 has two air purification phases and, as explained, below, each has specific power requirements that may differ from the other phases. Generally, air treatment system 10 includes an intense field filter phase comprising a pair of intense field dielectric units 32, each of which is associated with one of the air inlet 14 and operatively coupled thereto to filter any air drawn into housing 12 via inlets 14. Air treatment system 10 further includes a dielectric barrier discharge phase comprising dielectric barrier discharge unit 34 that is positioned to treat air within the air flow pathway of housing 12 after filtering by intense field dielectric units 32 by discharging plasma into the filtered air. Intense field dielectric units 32 and dielectric barrier discharge phase 34 combine to purify air passing through air treatment system 10 to remove contaminants, including biological hazards such as bacterial and viruses, as well as small particulate matter and chemical pollutants. The combination of intense field dielectric units 32 and a dielectric barrier discharge unit 34 synergistically ensure that any pathogens, including viruses, are inactivated or filtered out, thereby providing a significant safety improvement over conventional systems that rely on single purification phases such as UVC irradiation and allow for continuous use in a location with high throughput. As seen in FIG. 2, intense field dielectric units 32 of air treatment system 10 are positioned adjacently to inlets 14 so that air flow into passageway 16 must flow through intense field dielectric units 32.

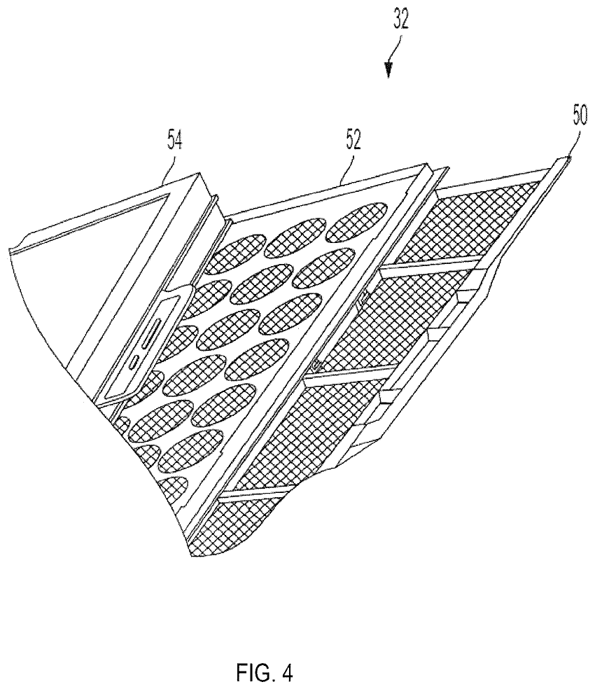

Referring to FIG. 4, intense field dielectric units 32 each comprises a prefilter 50, a field generator 52, and intense field dielectric filter 54 that are aligned for treatment of air in passageway 16 as it flows therethrough. Prefilter 50 comprises a conventional filtration panel having low resistance that can filter out particles having a size between 1 and 2 millimeters. Prefilter 50 is therefore intended to remove large airborne particles and debris from the air flow. Prefilter 50 is preferably washable for reuse and manufactured from materials that provide a long service life. The pre-filter 50 can be made of a nylon material or be a traditional paper type filter.

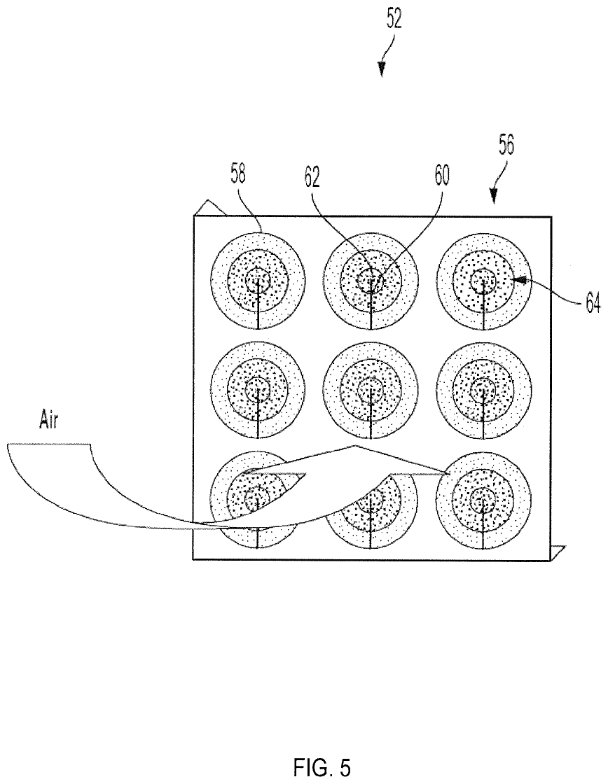

Referring to FIG. 5, field generator 52 comprises a thin metal plate 56 having a series of circular or square holes 58 extending through plate 56 and positioned in an array about the major surfaces of plate 56. A pin electrode 60 is positioned so that its tip 62 is located in the middle of each hole 58. The application of a voltage between the tip 62 of pin electrode 60 and the edge of hole 58 creates an effect referred to as a corona discharge 64 within the holes 58. As airborne particles in the air flowing through passageway 16 pass through the corona discharge 64 formed in hole 58, the airborne particles will become charged. The electrode 60 is between 0-50 mm from the edge of the hole 58. The field generator 52 transforms 24 volts of direct current (VDC) input to the 8000 VDC used to create the corona discharge.

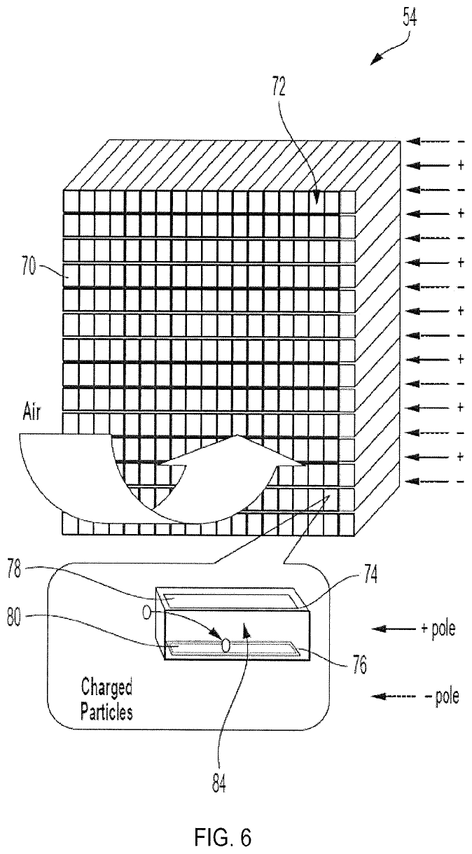

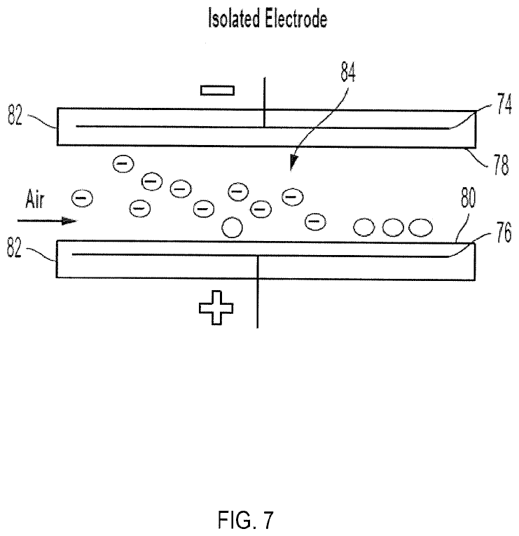

Referring to FIG. 6, intense field dielectric filter 54 comprises a grid 70 defining a plurality of microchannels 72. Grid 70 is positioned proximately to field generator and aligned therewith so that air flowing through holes 58 of field generator 52 will pass through microchannels 72. Microchannels 72 may have cross-sectional dimensions of approximately 3 mm by 1.2 to 1.5 mm or 3 mm by 1.7 to 2 mm. The depth of the microchannels 72 may vary as needed, but may be between 25 and 50 mm. Each microchannel 72 is formed by a pair of spaced apart electrodes 74 and 76 defining two opposing lateral surfaces 78 and 80 of microchannel 72 (depicted as the top and bottom surfaces of a rectangular channel, but it could instead be the left and right sides). Electrodes 74 and 76 are wrapped with dielectric material 82 which protects against electric shocks and increases the service life of filter 54. Every adjacent electrode 74 and 76 is oppositely charged, so that each microchannel 72 has one lateral surface 78 having a positive or negative charge while the opposing lateral surface 80 has the opposite charge, thereby forming a strong electric field within the space 84 formed inside each microchannel 72. The microchannel 72 utilizes 24VDC to create the electric field. Charged air particles leaving field generator 52 after being charged by corona discharge 64 will pass into microchannels 72 and enter the strong electric field formed therein. Any charged particles will be arrested and firmly held by an oppositely charged internal lateral surface 78 or 80 of microchannels 72, as seen in FIGS. 6 and 7. Microchannels 72 of intense field dielectric filter 54 have relatively low resistance and thus produce a minor pressure drop in air flow of between 10 and 30 Pascals. Intense field dielectric filter 54 can provide an arresting capability of close to 100 percent for charged particles passing through microchannels 72. For example, an exemplary system can reduce the concentration of atmospheric particulate matter of 2.5 micrometers (PM 2.5) from 999 micrograms per cubic meter (ug/m³) to 46 micrograms per cubic meter (ug/m³). Pathogens such as bacteria that are carried by arrested particles can be trapped and destroyed by the high strength electrical field. Under normal loads, intense field dielectric filter 54 can be used for up to a year before cleaning is needed. Even if intense field dielectric filter 54 is powered off, static electricity will remain in filter 54 for a long period of time to firmly lock any adsorbed dust on either of electrode 74 and 76. The dielectric filter 54 can be cleaned by using a vacuum with a brush attachment, and if necessary, a neutral cleaning agent and a soft brush and water.

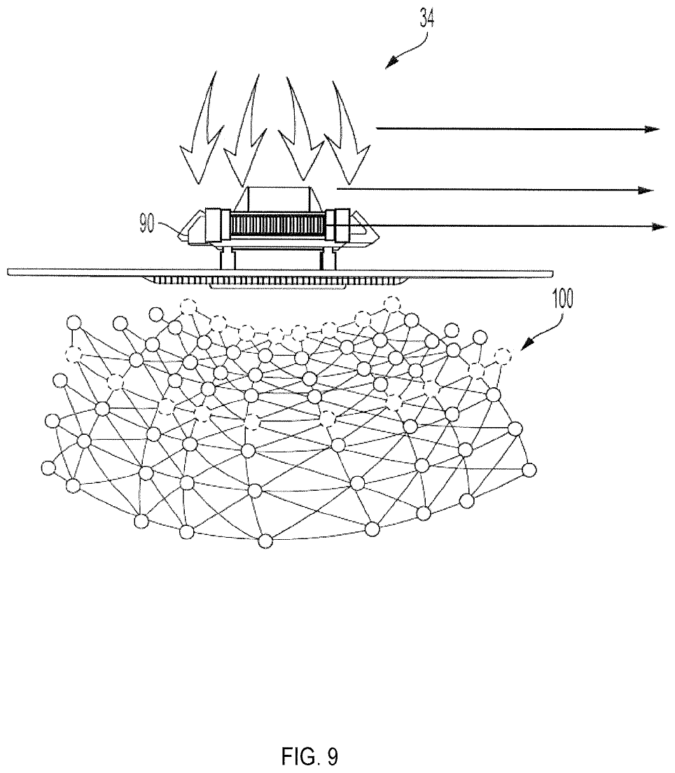

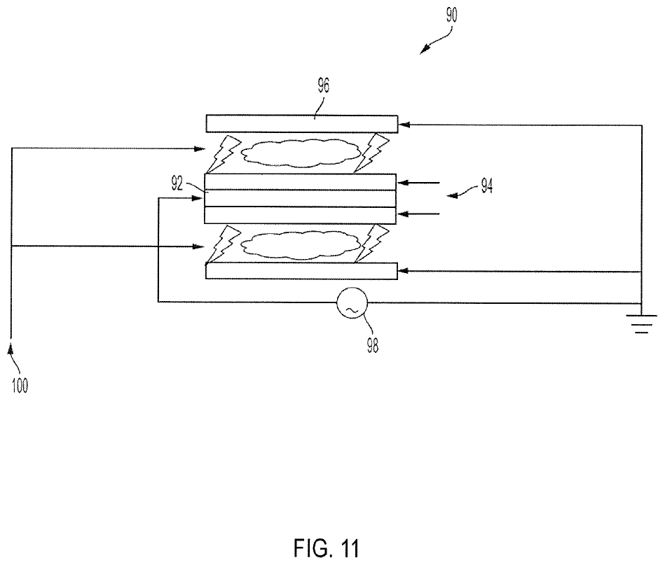

Referring to FIGS. 9 and 10, dielectric barrier discharge phase 34 comprises a dielectric barrier discharge unit 90 having a high voltage discharge electrode 92 coupled to a dielectric barrier 94 and spaced apart from a ground electrode 96. When a high voltage AC generator 98, such as one operating at 2800 VAC, is coupled to high voltage discharge electrode 92 and ground electrode 96, a large quantity of positive and negative oxygen ions are generated in the chamber 100 between high voltage discharge electrode 92 and ground electrode 96. Dielectric barrier discharge unit 90 thus produces a bi-polar ionized gas discharge in the discharge chamber 100. Dielectric barrier 94 can cover the electrode or be suspended in the discharge space 84. When a sufficiently high AC voltage is applied to discharge electrode 92, such as at 2800 VAC, the gas between the electrodes 92 and 96 will be broken down at a very high gas pressure to form what is referred to as a dielectric barrier discharge or low temperature plasma. By systematically controlling the electrode structure and discharge parameters, dielectric barrier discharge unit 90 can carry out discharge work in a relatively low voltage and produce free electrons with high potential and kinetic energy. In the discharge space 84, the atoms in the molecules gain enough kinetic energy to separate from each other or dissociate, with the outer electrons of atoms becoming free to produce ions. For example, dielectric barrier discharge unit 90 operating at 5 Watts can generate 3.5 to 5 million ions per cubic centimeter (cm³) for comprehensive and continuous purification without any secondary pollution i.e. no ozone is produced. The ions will attach to and break down any pathogens in the air, such as bacteria and viruses, as well as any chemicals, such as formaldehyde, TVOC, ammonia, and cigarette smoke residue. The ions additionally attach to small particulate matter which results in the coagulation of them due to charge polarity, resulting in increased weight and particulate dust dropping from the air. The ionic disinfection provided by dielectric barrier discharge phase 34 may continue as air leaves housing 12 and is passed into the enclosed location. Maintenance of dielectric barrier discharge phase 34 is limited to brushing of the surface periodic (three to six month intervals) to remove any accumulated dust.

System 10 may further comprise a local controller programmed to dynamically operate any one or more of intense field dielectric phase 32 and dielectric barrier discharge phase 34 according to current conditions. For example, it may be possible to determine the current quality of the air to determine real-time demands of the location so that system 10 is operated at maximum efficiency to ensure adequate air purification while reducing power consumption, extending the lifespan of the components, and maximizing service intervals. In addition, visual indicators may be used to indicate to consumers the status of system 10, such as whether air purification is active and fully operational. System 10 may contain an hour meter to display the number of operational hours the unit has been active for, in order to help dictate maintenance periodicity.

Intense field dielectric units 32 and a dielectric barrier discharge phase 34 work harmoniously to provide germicidal irradiation, physical filtration to remove particles and reduce virus transmission, and disinfection through the release of disinfection factors (positive and negative oxygen ions). The solution of the present invention thus can effectively reduce the infection risk and range of a pathogen such as a virus, while also serving as a mechanism for disinfection of the enclosed location.

The synergistic effects of the combination of intense field dielectric filters 32 and a dielectric barrier discharge phase 34 of system 10 were evaluated and demonstrated with respect to removing/eliminating aerosolized MS2 Bacteriophage ATCC 15597-B1, as well as E. coli ATCC K-12. The efficiency of the device in an aerosol test study was evaluated to determine the effectiveness of the device to eliminate COVID-19. The efficacy of system 10 to eliminate aerosolized viruses in ISO 17025 accredited United States based laboratory testing in compliance with the EPA and FDA guidelines. Accordingly, two sets of testing were completed to validate the efficacy of system 10 to indicate personnel protection against COVID-19 and against other various viruses, bacteria, and hazardous airborne particulates.

In a first test, system 10 comprised intense field dielectric filter 32 and dielectric barrier discharge phase 34, with a text box and cabling for actuation of the individual subsystems. The unit included recirculated and supply air sections to demonstrate system 10 integrated into a baseline representative model. System 10 was tested using 15, 30 and 60 minute contact times with the MS2 bacteriophage ATCC 15597-B1. A first set of testing at the longer contact times was intended to provide validation results in a comparable format with other products, which were tested under similar parameters. Six total test runs were performed in single replicate for device runs and triple replicate aerosolized sample collection to evaluate efficacy to remove/inactivate the MS2 bacteriophage ATCC 15597-B1 from the air, including a control run and various combinations of the devices.

The MS2 was first inoculated and then aerosolized into the test chamber via nebulizers for 60 minutes to reach appropriate concentration, then baseline samples were taken at t=0 min, and additional samples were then taken at t=15 min, t=30 min, and t=60 min. After the samples were collected, they were plated and incubated and then enumerated to determine microbial concentration. Additional testing at shorter contact times was carried out using the MS2 and E. Coli in single replicate sampling at 1, 3, and 5 minutes. In total, 6 test runs were performed again under a similar process as described above.

The testing was performed with MS2 Bacteriophage, which is a small, non-enveloped virus that is recognized by the EPA as one of the most difficult type of viruses to inactivate and therefore considered by the EPA to be a representative viral screening tool. Specifically, there is a hierarchy that is generally applied to categorize these, which includes: (1) Small, non-enveloped viruses—most difficult to inactivate (MS2 Bacteriophage fits in this categorization) e.g. poliovirus, enterovirus, or rhinovirus; (2) Large, non-enveloped viruses—moderately difficult to inactivate e.g. adenovirus, rotavirus, or papillomavirus; and (3) Enveloped viruses—easy to inactivate (COVID-19 fits this categorization) e.g. influenza, herpes virus, or hepatitis virus.

For all runs, 0.5 ml of MS2 bacteriophage ATCC 15597-B1 stock and 10.0 ml of E. coli ATCC K-12 culture were added to 34.5 ml of Phosphate Buffered Saline and mixed until homogeneous. 20.0 ml of inoculum was added to each nebulizer. MS2 virions are 23-28 nm in diameter and non-enveloped, compared to the COVID-19 virus, which is 60-140 nm in diameter and enveloped. Therefore, it is harder to capture the MS2, more difficult to irradiate in terms of surface area, and requires significantly more radiation to inactivate. On this predication of the testing and its relevance for the intended application, the results presented can be construed to represent the minimum efficacy against COVID-19 and other flu-like viruses.

Air samples were taken in single replicate at the following time points after the device was running: 1 minute, 3 minutes and 5 minutes. Device was turned off after 5 minutes of total treatment time and samplers were allowed to continue sampling. Test microorganisms were grown on appropriate media. Cultures used for test inoculum are evaluated for sterility, washed and concentrated in sterile phosphate buffered saline upon harvesting. The test inoculum was split into two equal parts and added to the appropriate number of nebulizers. Liquid culture did not exceed 20 ml per nebulizer. The device was setup per protocol requirements and operated per manufacturer's instructions. The chamber is setup and the safety checklist was completed prior to test initiation. Test was initiated by aerosolizing the microorganisms per the nebulizers and allowing the concentration to reach the required PFU/m³. Once the concentration was reached, a time zero sample was taken, then the device was operated for the specified contact time and an additional sample was taken for each contact time. A decontamination process was run, 4 hours of UV exposure, prior to any humans entering the testing chamber. Samples were enumerated using standard dilution and plating techniques. Microbial concentrations were determined after appropriate incubation times. Reductions of microorganisms are calculated relative to concentration of the time zero or corresponding control run sample as applicable.

System 10 achieved a significant reduction in the aerosolized virus in a very short time interval. After just five minutes, the system reaches 99.98%, which is approximately a sanitation level equivalent to using standard hand sanitizer (99.99%). At 15 minutes, system 10 achieved a 99.99993% elimination of virus, approaching sterilization levels as it is increasing to a >99.99998% reduction after 30 minutes, and continuing >99.99998% through the 60-minute test period. Additional testing was performed as E. Coli at the shorter contact times to provide another live microorganism example to demonstrate the efficacy against bacterium. The system reached up to 99.998% efficacy as soon as one minute. The result demonstrate that system 10 acts to filter and purify the air and is effective not only on the immediate threat of the COVID-19 virus but that it will provide the same level of protection against other virus that recur annually in cold and flu season.

Table 1 below provides the test results for MS2 Bacteriophage ATCC 15597-B1 and system 10 with intense field dielectric filter 32 and dielectric barrier discharge phase 34.

TABLE 1

| | | Percent | Adjusted | Log₁₀ | Adjusted |
|---|---|---|---|---|---|
| | | Reduction | Percent | Reduction | Log₁₀ |
| | | Compared | Reduction² | Compared | Reduction¹ |
| Time | Average | to Time | Compared | to Time | Compared |
| (mins) | PFU/m³ | Zero | to Baseline | Zero¹ | to Baseline |
| 0 | 1.13E+09 | N/A | N/A | N/A | N/A |
| 15 | 3.10E+02 | 99.99997% | 13.196% | 6.56 | 5.68 |
| 30 | 3.81E+02 | 99.99997% | 11.797% | 6.47 | 5.54 |
| 60 | <2.35E+02 | >99.99998% | >4.996% | >6.68 | >5.38 |

The limit of detection for this assay is 8.00+01 PFU/m3 and values below the limit of detection are noted as “<8.00E+01” in the data table.
¹The Log reductions for the Test Runs are adjusted to account for natural die-off and gravitational settling observed in the Control Run.
²The Percent reductions for the Test Runs are adjusted to account for the natural die-off and gravitational settling observed in the Control Run.

Table 2 below provides the detailed test results for MS2 Bacteriophage ATCC 15597-B1 and system 10 with shorted contact times:

TABLE 2

| | | Percent | Log₁₀ | Adjusted |
|---|---|---|---|---|
| | | Reduction | Reduction | Log₁₀ |
| | | Compared | Compared | Reduction¹ |
| Time | | to Time | to Time | Compared |
| (mins) | PFU/m³ | Zero | Zero | to Baseline |
| 0 | 1.02E+09 | N/A | N/A | N/A |
| 1 | 4.01E+06 | 99.61% | 2.41 | 2.34 |
| 3 | 7.30E+05 | 99.93% | 3.15 | 3.08 |
| 5 | 4.06E+05 | 99.96% | 3.40 | 3.23 |

The limit of detection for this assay is 8.00+01 PFU/m³ and values below the limit of detection are noted as “<8.00E+01” in the data table.
¹The Log reductions for the Test Runs are adjusted to account for natural die-off and gravitational settling observed in the Control Run.

Table 3 below provides the detailed test results for E. coli ATCC K-12 and system 10.

TABLE 3

| | | Percent | Log₁₀ | Adjusted |
|---|---|---|---|---|
| | | Reduction | Reduction | Log₁₀ |
| | | Compared | Compared | Reduction¹ |
| Time | | to Time | to Time | Compared |
| (mins) | CFU/m³ | Zero | Zero¹ | to Baseline |
| 0 | 4.71E+06 | N/A | N/A | N/A |
| 1 | <8.80E+01 | >99.998% | >4.73 | >4.65 |
| 3 | <8.80E+01 | >99.998% | >4.73 | >4.65 |
| 5 | <8.64E+01 | >99.998% | >4.74 | >4.65 |

The limit of detection for this assay is 8.00+01 PFU/m3 and values below the limit of detection are noted as “<8.00E+01” in the data table.
¹The Log reductions for the Test Runs are adjusted to account for natural die-off and gravitational settling observed in the Control Run.

By deploying system 10, it is possible to reduce intense cleaning regimes that have been put in place and provide an independently validated filtration and purification system that will begin to restore confidence and encourage the use of indoor locations.

What is claimed is:

1. An air purification system, comprising:
a housing having an inlet, an outlet, and a passageway extending between the inlet and the outlet within the housing to define an air flow pathway;
at least one fan positioned in the housing to create and maintain a pressure differential along the passageway such that air can flow into the inlets, through the passageway, and out of the outlet along the air flow pathway;
an intense field unit coupled to the inlet, wherein the intense field unit comprises an intense field generator having a series of openings formed therethrough and a corresponding series of electrodes positioned in each of the series of openings so that a tip of each electrode extends into a center of each opening respectively, and an intense field dielectric filter having a plurality of channels formed therethrough and aligned with the openings of the intense field generator, wherein each channel is defined by a first surface comprising a first electrode and a second surface opposing the first electric and comprises a second electrode, and wherein the first electrode and the second electrode are encompassed by a dielectric material; and
a dielectric barrier discharge unit having a high voltage electrode that is coupled to a dielectric barrier, a ground electrode spaced apart from the high voltage electrode to define a low temperature plasma discharge chamber, wherein the discharge chamber is in communication with the passageway.

2. The air purification system of claim 1, further comprising a first power source coupled to the intense field generator to apply a first voltage to the tip of each electrode and to an edge of each opening that is sufficient to create a corona discharge therebetween.

3. The air purification system of claim 2, wherein the voltage applied to the tip of each electrode and an edge of each opening is about 8000 volts of direct current.

4. The air purification system of claim 3, further comprising a second power source coupled to the intense field dielectric filter to apply a second voltage to the first electrode and the second electrode.

5. The air purification system of claim 4, wherein the second voltage is 24 volts of direct current.

6. The air purification system of claim 5, wherein the channels are configured to result in a pressure drop in the air flow path of less than about 30 Pascals

7. A method of purifying the air in a location, comprising the steps of:
positioning an air treatment unit in the location, wherein the air treatment unit includes a housing having an inlet, and an outlet, and a passageway extending between the inlet and the outlet to define an air flow pathway, at least one fan positioned in the housing in the passageway, an intense field dielectric unit associated with the inlet, and a dielectric barrier discharge unit in communication with the passageway;
operating the fan to create and maintain a pressure differential along the passageway so that air flows from the location into the inlet, along the passageway, and out of the outlet into the location;
powering the intense field generator to create a corona discharge;
powering the intense field filter to capture any particles in the air that flows through the passageway that are charged by the corona discharge; and
powering the dielectric barrier discharge unit to emit low temperature plasma into the passageway.

8. The method of claim 7, wherein the step of powering the intense field generator comprises applying a first voltage to the tip of each electrode of a plurality of electrodes and to an edge of each opening of a corresponding plurality of openings that surround the plurality of electrodes to create a corona discharge therebetween.

9. The method of claim 8, wherein the voltage applied to the tip of each electrode and the edge of each opening is 8000 volts of direct current.

10. The method of claim 9, wherein the step of powering the intense field dielectric filter comprising applying a second voltage to a first plurality of electrodes and a second plurality of electrodes that are spaced apart by a plurality of microchannels.

11. The method of claim 10, wherein the second voltage is 24 volts of direct current.

12. The method of claim 11, wherein the step of powering the dielectric barrier discharge unit comprises supplying 2800 volts of alternating current to a high voltage electrode of the dielectric barrier.
